(19) United States
(12) Patent Application Publication
(10) Pub. No.: US 2009/0149771 A1
(43) Pub. Date: Jun. 11, 2009

(54) Vessel locator

(75) Inventors: Helge Myklebust, Stavanger (NO); Lance Becker, Philadelphia, PA (US); Bob Neumar, Wayne, PA (US); Tore Laerdal, Stavanger (NO); Kenneth George Morallee, Orpington (GB); Vinay Nadkarni, Media, DE (US); Joshua Lampe, Philadelphia, PA (US); Raina Merchant, Philadelphia, PA (US)

Correspondence Address: Karen Lenaburg, Esq.;DORSEY & WHITNEY LLP, Suite 3400, 1420 Fifth Avenue, Seattle, WA, 98101, US

(73) Assignee: Laerdal Medical AS, Stavanger (NO)

(21) Appl. No.: 11/999,740

(22) Filed: Dec. 5, 2007

(51) Int. Cl. A61B 5/053

(52) U.S. Cl. 600/547

(57) ABSTRACT

The present invention is a vessel detector comprising a sensor, a display and an access device for positioning the sensor in a detection area. According to the invention the sensor device is an impedance sensor. The invention comprises also a method for locating vessels comprising impedance measuring.

### TECHNICAL FIELD

The present invention is directed to devices and methods for determination of blood vessel location.

### BACKGROUND OF INVENTION

One of the most frequently performed medical procedures is the insertion of a needle into a live human body for the purpose of drawing blood from a vessel, delivery of fluids and drugs, inserting a catheter, performing diagnostic tests, administering medications, etc. Despite the frequency of this procedure, accurate needle insertion is often difficult due to difficulty in locating the desired vessel. Factors confounding vessel location include, but are not limited to, low or no blood pressure (elderly, cardiac arrest), the vessels being small (children), or the vessels not being easily visualized or palpated (obesity, tissue damage).

In cardio pulmonary resuscitation it can be necessary to provide vascular access. Perhaps the most effective way of getting vascular access in the emergency situation is to “cut down” and expose the vessels typically in the femoral or jugular site. But this procedure is felt to be messy, risky and time consuming. For those who are well trained and experienced, it could just take 5 minutes to get access, but the traditional emergency physician would need much longer. There are also additional concerns about causing unnecessary harm while performing the cut down procedure.

Over the years, minimally invasive surgical procedures have become more and more used. Now surgeons only need a few small holes to perform complicated surgical operations. To provide a clear field of view for an endoscope, gas or clear fluids are connected to get better access and fluids are circulated to clear blood from the field of view. This technique requires significant training to learn to efficiently use the surgical tools and to learn to recognize anatomical structures in a limited field of view.

Currently, several methods are being used to locate blood vessels. For instance, the use of anatomical landmarks to estimate the location of blood vessels based on position of visible features (articulations, muscles) and palpation of non-visible structures is a widespread technique in clinical practice. A clear disadvantage of this method is its low accuracy for certain patients (e.g. obesity patients, elderly) and certain medical situations (e.g. cardiac arrest).

Another method, known as “Popping”-detection, is also a widespread method for vessel location. This technique comprises inserting a needle in a body part at the site where a vessel is supposed to be. Because a vessel wall is elastic up to a certain degree, it is possible to notice a change in mechanical resistance to penetration when the needle perforates the vessel wall. This method also has several disadvantages. First, this method can require several attempts. Second, the vessel walls in elderly, children and cardiac arrest patients usually lack the elasticity necessary to ensure detection. Third, the use of gloves reduces the operator's sensation of popping as well as the ability to palpate non-visable structures.

Another method, “Flash back”-observation, is the observation of blood in the introduced needle when a blood vessel is perforated. This method has similar disadvantages as “popping”-detection.

U.S. Pat. No. 5,280,787 and U.S. Pat. No. 6,056,692 disclose ultrasonic scanning of a body part to locate blood vessels. This technique requires advanced equipment and is again subject to error due to reduced or non existent blood flow. Additionally, this method requires significant training to ensure competent use of the ultrasound device.

As one can see, techniques with an acceptable accuracy (landmarks, flush-back, popping) require trial and error performed by means of needles, which leads to lack of efficiency, delays, potential injury and patient discomfort.

Thus, there is a need for a vessel locator which provides swift vessel location with minimal injury and discomfort. The ideal system facilitates quick and safe vascular access, is perceived not to harm the patient and can also be easily learned and remembered.

### SUMMARY OF THE INVENTION

In one aspect of the invention, a vessel detector comprising a sensor, a display and an access device for positioning the sensor in a detection area. In one embodiment of the invention, the sensor device is an impedance sensor.

### BRIEF DESCRIPTION OF THE DRAWINGS

FIG. 1 is an illustration showing one embodiment of the invention.

FIG. 2A is cross section of a sense electrode and a vessel according to one embodiment of the invention.

FIG. 2B is a cross section of a sense electrode including a camera according to one embodiment of the invention.

FIG. 3A shows positioning of the vessel detector on a patient's leg according to one embodiment of the invention.

FIG. 3B shows different tissues and illustrates how a device will be introduced at an angle according to one embodiment of the invention.

### DETAILED DESCRIPTION

Embodiments of the present invention are directed toward a device and method for determining the location of blood vessels. Certain details are set forth below to provide a sufficient understanding of the embodiments of the invention. However, it will be clear to one skilled in the art that various embodiments of the invention may be practiced without these particular details.

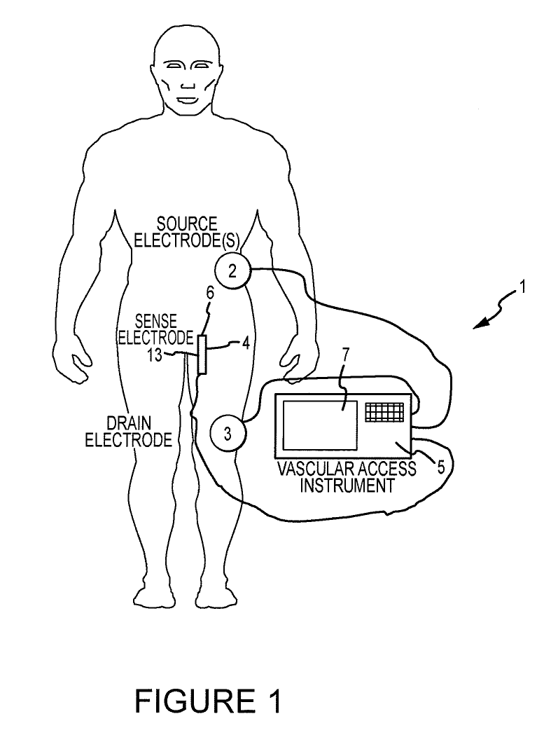

FIG. 1 shows a vessel detector 1 situated in the femoral vein of a patient and being part of a location system according to one embodiment of the invention. In this embodiment, the detector's impedance sensor device comprises one or several near constant current sources capable of delivering a few milliamps of alternating current. In case several current sources are provided, the frequency of the current can be different between the sources. In this embodiment, each current source connects to one source electrode 2 and 3, respectively. In another embodiment, a multiple electrode configuration may be used. Another embodiment includes using a single source electrode configuration. For instance, a single source configuration comprises one source electrode, such as either electrode 2 or electrode 3 connected to one constant current source 5 which can operate on multiple frequencies. A sense electrode 13 is arranged with a conductive area which is small comparable to the source electrodes 2 and 3. This much smaller conductive area will thus cause a much higher current density in a volume of tissue close to the active electrode area. Consequently, the impedance of the tissue within said volume will characterize the impedance seen between the two electrodes. The sensor or sense electrode 13 is in connection with an access device 4 for positioning the sensor in a detection area and is made out of a conductive and bio-compatible material like stainless steel.

When the tip 6 of device 4 is in contact with the skin surface, a very high impedance will be detected. In contact with fat, the impedance is still quite high, but smaller than skin impedance. In contact with skeletal muscle, the impedance is reduced markedly compared to fat. In contact with large blood vessels the impedance is as low as it gets. This is an indication that the tip 6 of the device 4 and thus the sense electrode is in contact with one large blood vessel. Smaller vessels may cause an artifact as the said volume of measurement in that case will include a combination of a small vessel and its surrounding tissue.

An article discussing impedance relative to body tissue is Tissue Resistivity (from Geddes, Baker: “Principles of applied biomedical instrumentation”, a Wiley-Interscience Publication. New York, 1989, chapter 11), incorporated herein in its entirety by reference.

| | |
|---|---|
| Blood | 150 Ω-cm |
| Plasma | 60 Ω-cm |
| Urine | 30 Ω-cm |
| Skeletal Muscle | 300 Ω-cm (longitudinal) |
| Skeletal Muscle | 1600 Ω-cm (transverse) |
| Fat | 2500 Ω-cm |
| Skin | 3000 Ω-cm at 1 MHz |

Display 7 is arranged to receive signals from the sensor and to display an indication of the presence of blood vessels. One such indication can be implemented as an image of the detection area. A visual image will be provided by detector 1 if the system comprises a camera.

In one embodiment of the invention, the tip 6 of device 4 comprises a fastening means to suction the vessel firmly in contact with the tip 6. This suction force is provided by a suction pump which can be integrated with the source 5, and where a suction fastening device is connected to device 4 using a flexible tubing. The suction force may be provided manually, electrically, by the use of compressed gas, or any other method capable of providing suction. Device 4 might be arranged with a number of suction channels to facilitate good contact with the vessel.

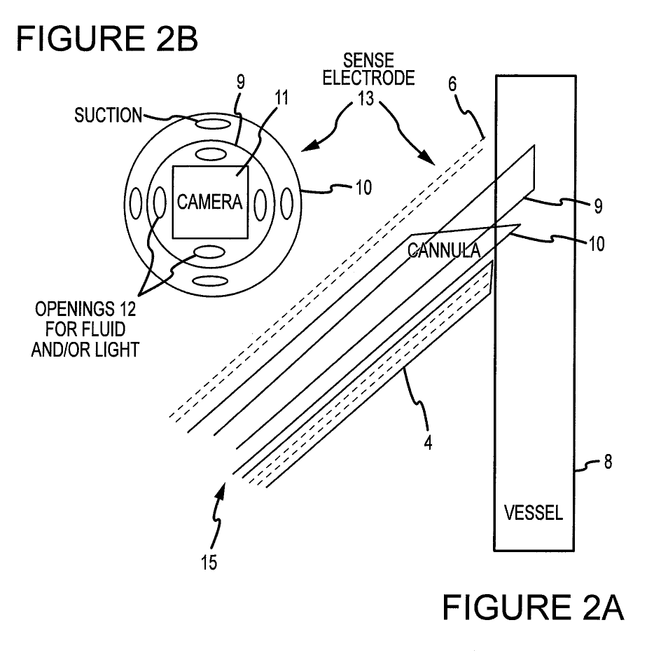

FIG. 2A shows a vessel 8 and a suction connection between access device 4 and vessel 8 according to one embodiment of the invention. When the vessel 8 is firmly secured to the access device 4 by vacuum forces, a cannula 15 can be inserted through the sensing electrode to penetrate the vessel 8. The cannula 15 is preferably arranged with an inside element 9 and an outside element 10 such that when these are forced against each other, they form a seal which helps the vascular entry to remain fluid tight. This is achieved when the inside element 9 and the outside element 10 have a diameter which is larger than the diameter of the hole through the vessel. The suction assists with fluid and/or blood removal.

In one embodiment, the cannula 15 has a small in diameter, approximately the size of a pencil, to facilitate penetration of the skin at the desired location. The jugular or femoral locations on the body are the most commonly used.

Even a small penetration can cause bleeding. For this reason and as will be discussed below, the detector can circulate clear fluid such that blood or other visual impediments can be washed from the site. The flushing fluid used has limited conductivity in order to support the impedance system, i.e. distilled water.

The access device or penetrating body can be further arranged with a vacuum tip, such that when the vessel is located by visual and/or impedance means, it can be connected to the tip of the cannula using vacuum in order to facilitate vessel penetration and vessel-cannula connection.

FIG. 2B shows one embodiment of the invention which comprises a small video camera 11, which can sit in the core of the cannula. The camera 11 may be an type of imaging device which permits visualization of the detection area to aid navigation to the vessel. In one embodiment, the camera 11 is a digital camera, such as a CCD camera or the like. In one embodiment, the diameter of the camera 11 body fits within the inner diameter 9 of the cannula 15. The camera 11 is connected to a power source, such as by cable or battery, and to display 7, such as wirelessly or by cable.

In one embodiment, when using a camera, the imaging device can comprise a transparent and electrically conductive layer in the area arranged for contact with a detection area.

In yet another embodiment of the invention, one or more light sources 12 are provided for the camera. In one embodiment, the light source is a set of LED arranged next to the camera. In another embodiment the light source is provided externally and a set of light fibers are used to bring the light to the tip of the sense electrode.

Because blood often reduces visibility, a fluid source might be necessary. The fluid can be arranged as part of the system in openings 12, coincident with the light sources or in separate openings 12. In addition, the fluid can be a simple bag of fluid that is left elevated from the access site.

In addition, because the sensor tip 6 is arranged with suction, it will also bring out fluid with blood and thereby enhance visibility. With the camera on the tip 6 of the access device 4, the operator can now use the display 7 of FIG. 1 to visually locate the vessels, before using the impedance signal for verification.

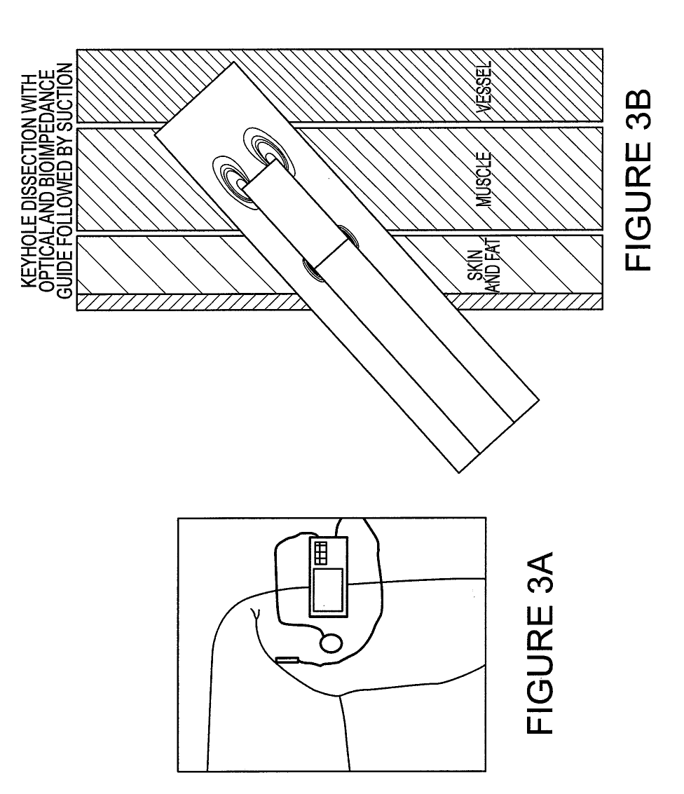

FIG. 3A illustrates positioning of one embodiment of the vessel detector on a patient's leg, for localisation of the large femoral vessels.

FIG. 3B shows different tissues and illustrates how the device according to one embodiment of the invention will be introduced to the tissue at an angle to the blood vessel.

As stated above, the vessel detector, thus uses impedance measurements to detect a blood vessel, using the principles of keyhole surgery, namely that the incisions in the skin are made as small as possible, and that necessary instrumentation is entered trough those incisions. Minimally invasive surgery uses a camera or fiber optics to allow visual identification of structures of interest. The vessel detector primarily uses impedance analysis to identify a structure, in this case a blood vessel.

Although the present invention has been described with reference to the disclosed embodiments, persons skilled in the art will recognize that changes may be made in form and detail without departing from the spirit and scope of the invention. Such modifications are well within the skill of those ordinarily skilled in the art. Accordingly, the invention is not limited except as by the appended claims.

What is claimed is:

1. An apparatus for detecting a blood vessel, comprising:
an impedance sensor at a distal end of the apparatus;
an output device electrically coupled to the impedance sensor and mechanically coupled to a proximal end of the apparatus, the output device being operable to output a signal indicating an impedance detected by the impedance sensor; and
a fastening device operable to secure the apparatus to a wall of the blood vessel in response to a value of the output signal corresponding to a specific impedance value.

2. The apparatus according to claim 1, wherein the impedance sensor comprises at least one electrode connected to a current source.

3. The apparatus according to claim 1, wherein the fastening device secures the device to the detection area by providing suction force to the wall.

4. The apparatus according to claim 1, further Comprising an imaging device to provide an image of the blood vessel.

5. The apparatus according to claim 4, wherein said imaging device is a camera.

6. The apparatus according to claim 4, wherein the imaging device comprises a transparent and electrically conductive layer in an area arranged for contact with the detection area.

7. The apparatus according to claim 4, further comprising a light source.

8. The apparatus according to claim 1, further comprising a fluid delivery device and a fluid suction device.

9. The apparatus according to claim 1, wherein the access device comprises a conductive body connected to the sensor.

10. The apparatus according to claim 1, wherein the access device comprises a conductive tip.

11. A method of detecting and securing a blood vessel of a patient, comprising:
inserting a device into a body part of the patient;
detecting an impedance; and
in response to detecting a specific impedance value or a change in impedance, applying suction to secure the device to the blood vessel.

12. The method of claim 11 further comprising after the device is secured to the blood vessel, inserting an access device into the blood vessel.

13. The method of claim 11 wherein the access device is cannula.

14. The method of claim 11 wherein the suction is applied by a plurality of channels at the proximal end of the device.

15. The method of claim 11 wherein the act of detecting an impedance comprises detecting an impedance more than once.

16. The method of claim 15 wherein the act of applying suction to secure the device to the blood vessel is in response to a specific change in impedance value.

17. An apparatus for detecting and accessing blood vessel, comprising:
an impedance sensor at a distal end of the apparatus;
an output device electrically coupled to the impedance sensor and mechanically coupled to a proximal end of the apparatus, the output device being operable to output a signal indicating an impedance detected by the impedance sensor;
a fastening device adjacent the impedance sensor and operable to secure the apparatus to a wall of the blood vessel; and
an insertion device adjacent the fastening device, the insertion device operable to be Inserted into blood vessel after the fastening device is secured to the wall of the blood vessel.
